(19) United States
(12) Patent Application Publication
(10) Pub. No.: US 2015/0005637 A1
(43) Pub. Date: Jan. 1, 2015

(54) TISSUE DISPLACEMENT ESTIMATION BY ULTRASOUND SPECKLE TRACKING

(71) Applicant: UVic Industry Partnerships Inc., Victoria (CA)

(72) Inventors: Kelly Stegman, Victoria (CA); Nikolai Dechev, Victoria (CA); Slobodan Djurickovic, Victoria (CA)

(73) Assignee: UVic Industry Partnerships Inc.

(21) Appl. No.: 14/318,271

(22) Filed: Jun. 27, 2014

Related U.S. Application Data
(60) Provisional application No. 61/841,156, filed on Jun. 28, 2013.

(51) Int. Cl. A61B 8/08

(52) U.S. Cl. CPC A61B 8/5223; A61B 8/08; A61B 8/5253
USPC 600/449

(57) ABSTRACT

Tissue displacements are estimated with speckle tracking in B-scan images. A template region in a first image is compared with a plurality of image portions in subsequent image, and a tissue displacement is based on the comparison. In some examples, the comparison is based on a Fisher-Tippet distribution.

### CROSS REFERENCE TO RELATED APPLICATION

This application claims the benefit of U.S. Provisional Application No. 61/841,156, filed on Jun. 28, 2013, which is incorporated herein by reference.

### BACKGROUND

Tissue tracking techniques for clinical and laboratory applications tend to be complex and expensive. In addition, some methods require specialized hardware and cannot be adapted to conventional ultrasound systems. Conventional methods typically require operator trial and error, and are ill suited for unskilled operators. In most cases, ultrasound data acquired is converted for display purposes, making tissue tracking more difficult. Accordingly, improved methods and apparatus for tissue tracking are needed.

### SUMMARY

In some examples, methods of estimating a tissue displacement comprise selecting a template region in a first ultrasound image of a region of interest, wherein the first ultrasound image exhibits speckle. A plurality of image portions in a second ultrasound image of the region of interest are compared to the template region, wherein the second ultrasound image exhibits speckle. Based on the comparisons, a tissue displacement is estimated. In typical examples, the comparisons are based on a Fisher Tippet distribution or a Rayleigh distribution. In further examples, the first and second images are B-scan images, and total tissue displacement is established based on comparisons of image portions of a series of B-scan images to the template region. In other alternatives, the first and second images are RF envelope images, and a total tissue displacement is established based on comparisons of image portions of a series of RF envelope images to the template region. In some embodiments, a template region location is determined based on a displacement field associated with at least two ultrasound images. In yet other examples, a skip factor associated with a number of images between the first ultrasound image and the second ultrasound image is determined, and a template region size is based on an estimated image to image displacement and an image acquisition rate.

Representative apparatus comprise a memory configured to store a plurality of ultrasound images and a processor that receives the images from the memory, selects a region of interest and a template region in a first image, compares image portions in each of the series of images with the template region, and provides a tissue displacement based on the comparison. In some examples, the processor establishes the comparison based on a Fisher Tippet distribution and image values correspond to logarithmic functions of scattering amplitudes. In some examples, the images are B-scan images and the processor sequentially compares image portions in the series of images. In typical examples, the processor compares images in the series of images based on a skipping number associated with a number of images to be skipped between comparisons, wherein the skipping number is based on an expected lateral displacement per sequential image and a lateral resolution. In some embodiments, image segmentation is applied to at least one image to identify a specimen feature of interest, and a template region dimension is based on a dimension of the specimen feature of interest in the at least one image. Typically, the template region dimension is between about 30% and 80% of the specimen feature dimension, and the specimen feature of interest is a tendon. In one example, the processor provides the comparison based on maximization of

$$p ( \tilde{a} \vert \tilde{b} , \tilde{d} ) = {\prod }_{j = 1}^{IJ} \frac{2 exp 2 ( {\tilde{a}}_{j} - {\tilde{b}}_{j} )}{{[ exp 2 ( {\tilde{a}}_{j} - {\tilde{b}}_{j} ) + 1 ]}^{2}} ,$$

wherein ãⱼ and \tilde{b}ⱼ are elements of vectors of B-Scan intensities in the template region and series of image regions in each of the series of images.

Computer readable medium are provided that contain computer-executable instructions for performing a method comprising defining a template region in a selected image frame based on an image resolution, a specimen displacement between the selected image frame and an adjacent image frame, and an image feature size. An image portion in the template region in the selected image frame is compared with a plurality of test regions in a different image frame, and, based on the comparison, an image feature displacement is estimated. In some examples, the comparison is based on a Fisher-Tippet distribution.

These and other features and aspects of the disclosed technology are set forth below with reference to the accompanying drawings.

### BRIEF DESCRIPTION OF THE DRAWINGS

FIG. 1 illustrates a region of interest (ROI) within an image of a flexor digitorum superficialis (FDS) tendon. The tendon boundary is shown as a dotted boundary within image frame t+1, and is searched with TempBoxes such as a box ‘B’. Once a match is found, an interframe displacement vector is calculated as a difference in position between the Template (labeled ‘T’) from a previous frame t and a matching TempBox in frame t+1. The TempBox and Template have dimensions I by J, and the ROI has dimensions A by B.

FIG. 2 is a flow chart illustrating a method of estimating interframe displacement. After all Fisher-Tippett (FT) coefficients from all TempBox comparisons are stored, the TempBox comparison with the maximum FT value is considered the match and interframe displacement is calculated.

FIG. 3 illustrates a method associated with a fixed ROI method. FIG. 3(a) shows a frame t in which a Template (labeled ‘T’) is located at x₁,z₁. FIG. 3(b) shows an image frame t+1 in which a ROI is centered on the Template. A matching TempBox inside the ROI is found and the interframe displacement is calculated. This process is repeated: FIG. 3(c) shows a Template located at x₁,z₁ in frame t+1, and FIG. 3(d) shows a ROI in Frame t+2 centered on the Template location. A matching TempBox is found within the ROI, so that an interframe displacement can be calculated. The white disc in (a)-(d) is on top of the same area on the tendon, showing how the tendon displaces across the image frames as time increases.

FIG. 4 illustrates methods associated with interframe and total displacement processes using a fixed ROI or gating technique. A frame number t is incremented until a last or final frame of interest is reached. Interframe displacements from each comparison are added cumulatively to determine total displacement.

FIG. 5 illustrates a representative method of determining a template location.

FIG. 6 illustrates a representative method of determining specimen displacements using a displacement field.

FIG. 7 illustrates a representative method of determining a frame skipping factor.

FIG. 8 illustrates a representative apparatus for tissue tracking based on ultrasound speckle.

FIG. 9 illustrates a representative method of selecting a template size.

### DETAILED DESCRIPTION

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus' are referred to as “lowest”, “best”, “minimum,” or the like. It will be appreciated that such descriptions are intended to indicate that a selection among many used functional alternatives can be made, and such selections need not be better, smaller, or otherwise preferable to other selections.

As used herein, an ultrasound image generally refers to a two or three dimensional image of a specimen based on application of ultrasound. Such images can be displayed images, or numerical representations that are stored or storable in computer readable media such as RAM, ROM, CDs, hard disks, or other storage devices. Specimen images are generally obtained as a series of images, each of which can be referred to as a frame or an image frame. A next frame is a frame obtained directly following a prior frame, but in some examples discussed below, some frames are skipped. For convenience, the terms frame and image are both used in the following disclosure.

The disclosure pertains generally to speckle tracking-based methods to measure (quantify) internal 2-dimensional musculoskeletal (MSK) tissue displacement and velocity, using ultrasound-based imaging. In some examples, real time measurements are available. Some embodiments are focused on implementing speckle tracking methods that are computationally easy and fast, and therefore can be easily implemented on existing ultrasound hardware. This allows the proposed methods to be cost-effective software “add-ons” to existing machines, which can be easily used by clinicians. The disclosed technology has important applications in at least four areas: (a) in diagnostics, to help doctors determine muscle-tendon related impairment, (b) in surgical planning, (c) in assessment, by evaluating post-surgical outcomes and monitoring the post-surgical rehabilitation, and (d) in training researchers, technicians and resident doctors.

### Diagnosis

The disclosed methods can assist in the diagnosis of trauma to the muscle-tendon system by quantifying the MSK excursion. Typical causes of non-visible MSK trauma can include lifting heavy objects, blunt trauma and sports injuries. Patients with these injuries, particularly to the tendons, are often difficult to diagnose because the afflicted area will be in a painful and swollen condition. The assessment is often done in an emergency room (ER) or a GP office, where the need for internal visualization coupled with limited experience, makes diagnosis difficult. In the case where MSK tendon injuries which have torn from the insertion, are lacerated or ruptured, successful diagnosis is essential since the tendons must be repaired or re-attached. Failure to reattach tendons within 2-3 months will result in permanent functional loss of that tendon-muscle unit, due to muscle atrophy. Due to the ready implementation of the disclosed methods, many clinics could be available with little/no wait time for such assessments. A technician can use the disclosed methods and apparatus and ask a patient to attempt a series of finger flexions. The system can identify regions of interest, measure excursion as the patient flexes/extends as instructed, and create a report for further investigation by a radiologist in order to diagnose the rupture.

### Surgical Planning

In some cases, a surgical procedure known as muscle-tendon transfer is required to restore lost function. Tendon transfer becomes necessary when the muscle connected to the afflicted tendon has completely atrophied and become paralyzed. This may be due to delay in seeking medical help or delay in diagnosis. Furthermore, muscles affected by degeneration or nerve injury can also atrophy. In these cases of muscle atrophy or paralysis, surgical intervention known as muscle-tendon transfer can be used. The operation takes a redundant or less-needed tendon-muscle pair, cuts it from its original location, and uses it to substitute the damaged tendon-muscle pair. This way, the healthy muscle can perform the tendon action at the new location. The disclosed surgical planning methods can be used to identify the best donor tendons suitable for transfer, by estimating the excursion of the candidate donor tendons. Identifying the best tendon with similar excursion properties to the injured tendon, can be done by the surgeon prior to the operation, to help choose an ideal donor tendon. Previously, the selection of a non-ideal tendon would result in limited finger mobility due to tendon slack or over-tightness, which results in a need for additional corrective surgeries. Since surgical protocol is often surgeon-specific, and patients are individualistic, these methods may help standardize this procedure.

Rehabilitation with Post-Surgical Assessment

After surgical or non-surgical treatment of MSK injuries, the patient often undertakes a rehabilitation regimen. One way to measure rehabilitation success of tendon injuries is to quantify the degree of tendon displacement. Presently, such assessment is done by the therapist who measures the finger-joint rotation angles while they are flexed and extended, and also measures various dimensional parameters of the finger joints. All of this measured data is then used with one of three hand biomechanical models developed by Landsmeer. However, the accuracy of the Landsmeer models has been debated and there is a lack of consensus on which model best predicts tendon displacement. Alternatively, the proposed method provides a quick and direct measurement of tendon excursion. This can be measured multiple times throughout the rehabilitation regime in order to assess the effectiveness of treatment. In cases where finger mobility remains limited or less than expected during rehabilatation, the disclosed methods and apparatus can be used to diagnose the problem. Specifically, suture failure (tendon gapping or detached tendons), or slack tendons can be identified. Presently, without the disclosed approach, when evaluating a post-surgical patient with restricted finger mobility, or very limited flexion (rotation), it can be very hard to know what is causing the problem. For example, if the finger mobility is limited, it is hard to determine if the suture actually failed (which means a slack tendon, or suture failure), or if there is scarring around the tendon that is impeding the tendon motion. It is hard to differentiate between these two conditions externally, even by a specialist. The methods allow for non-invasive assessment and diagnosis of these issues, thus preventing the need for other invasive exploratory procedures. This can relieve additional healthcare costs and pressure on the healthcare system by using readily available ultrasound-based technology.

### Training Tool

Medical professionals such as researchers, resident doctors and technicians may require additional training with MSK functional anatomy. Since the disclosed methods can estimate MSK displacement using B-Scan ultrasound, these professionals can more easily diagnose MSK issues, and may also verify or develop biomechanical models involving muscle-tendon excursion.

### Ultrasound Image Speckle and Speckle Tracking

Ultrasound B-Scan images, rendered by the reflected soundwave from bone and tissues, are characterized by a granular appearance. This structure is often described as speckle texture, and is analogous to optical speckle phenomena observed with lasers. Speckle arises from the constructive and destructive interference pattern from the underlying scattering medium and is inherent to ultrasound imaging. Even though the observed speckle pattern does not correspond directly to the underlying tissue, the intensity of the speckle pattern reveals information on the local tissue. In particular, the speckle texture of tendons appears linearly striated and unidirectional, which is in contrast to the surrounding soft tissue. Ultrasonic speckle itself is usually considered a form of noise, causing image degradation. However, tracking the motion of speckles is a useful tool to detect tissue displacement in the absence of visual landmarks, which is often the case with tendons. As such, speckle tracking is a widely used method to estimate interframe (one image frame to a subsequent frame, often a next frame) displacement.

Several methods are disclosed herein that can track speckles in order to estimate MSK displacement in a sequence of consecutive ultrasound images. A representative disclosed method estimates MSK displacement based on a sequence of B-Scan ultrasound images using a block matching technique. The block matching technique defines a template sub-section in a reference ultrasound image frame. This template sub-section encompasses the desired section of speckle that is to be tracked, and the block matching method searches for a matching block in the subsequent frame. The criteria for determining a suitable match to the template in the subsequent frame utilizes a similarity measure as a comparison metric, called Fisher-Tippett (FT). Once the match is found, the interframe displacement is calculated. The following sections describe representative templates and regions of interest, how the templates are selected and compared to the blocks in the next or subsequent frames, how the similarity metric is derived, and how tracking is performed throughout the MSK's entire displacement.

### Templates and Regions of Interest

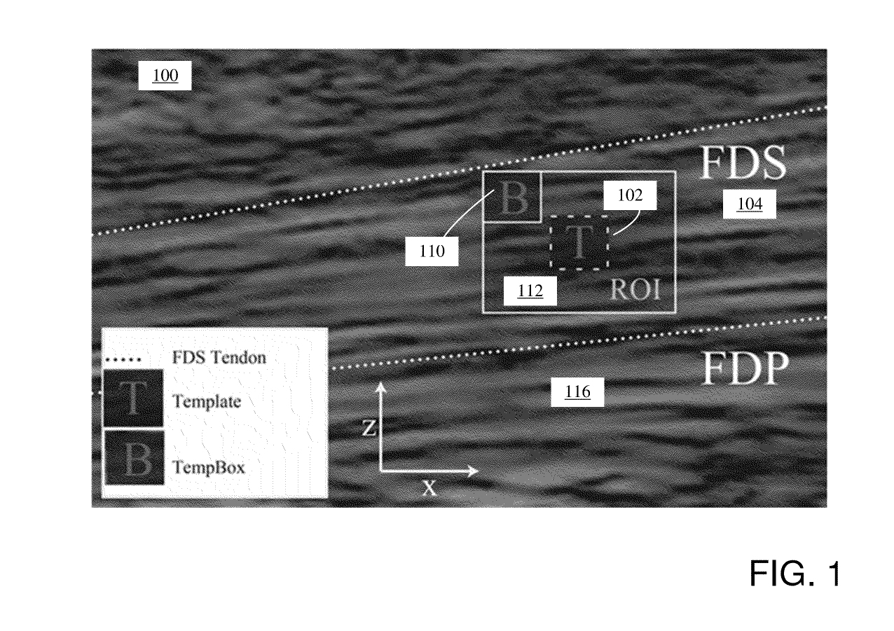

A B-Scan ultrasound image taken at time t consists of a 2-D array containing pixels, where each pixel has a grayscale intensity value. These intensities are numerically valued between, for example, zero and 255, and represent the intensity value of the reflected soundwave of the MSK tissue. To track the tendon displacement between frame t and frame t+1, a template is defined. A template is generally a data block of size I by J pixels, where I is a number of pixels along a first axis, such as an x (width axis), and J is a number of pixels along a second axis, such as a z (height axis) that is perpendicular to the first axis. In other examples, templates can be based on other sets of pixels such as areas of other shapes (rectangular, hexagonal, elliptical, or other regular or irregular shapes, including one dimensional arrays, and pixels along one or more non collinear axes can be used. As shown in FIG. 1, a template 102 is superimposed on a B-scan image 100 that includes a portion 104 corresponding to at least a part of an FDS tendon. The template 102 is located at x₁,z₁ on the B-Scan image frame 100 of the MSK tissue associated with a time t (referred to generally as a frame t). A B-Scan frame associated with a time t+1 is obtained, and searched to identify a block that matches the template 102 defined in image frame 100 at time t. The blocks to be considered as a potential match in frame t+1 are referred to as TempBoxes, and lie within a region of interest (ROI) with dimensions A by B, centered around x₁,z₁. A representative TempBox 110 is illustrated in FIG. 1. As shown in FIG. 1, TempBoxes and templates are generally defined within a region of interest (ROI) 112. A portion 116 of the image frame 100 is associated with a flexor digitorum profundus (FDP) tendon.

### Similarity Metric: Fisher-Tippett

The template in frame t is compared to several TempBoxes in frame t+1. Each comparison is made with the use of a similarity measure in order to quantify which TempBox in the ROI is the best match to the template. Typically, the Rayleigh (and FT) technique is used as a similarity measure for calculating the maximum likelihood that the template in frame t and a TempBox in frame t+1 are matched to each other. A similarity metric is calculated for each TempBox in the ROI. This section derives a similarity metric used for such a method.

In order to display the reflected soundwave from tissues in 2D, reflected signal strength is typically subjected to a compression process to form a B-Scan image. The pre-compression data, known as the RF-envelope-detected data, has a high dynamic range and cannot be properly displayed in this form. Speckle in an ultrasound RF envelope detected frame has been shown to follow a Rayleigh distribution. This means that if all the intensities in the RF frame were used to populate a histogram, the data would be Rayleigh distributed. Assuming that α=[α₁, α₂, . . . , αⱼ] is a vector of all intensities in the template in frame t and β=[β₁, β₂, . . . , βⱼ] is a vector of all intensities in a TempBox in frame t+1, wherein j is the total number of pixels in the template and TempBox. Given that a and b have respective Rayleigh distributed noise n₁ and n₂, the probability density functions (pdfs) p₁(n₁), and p₂(n₂) can be written as:

$$\begin{matrix}{p}_{1} ( {n}_{1} ) = \frac{{n}_{1}}{{σ}^{2}} exp ( \frac{- {n}_{1}^{2}}{2 {σ}^{2}} ) & \{ 1 \} \\ {p}_{2} ( {n}_{2} ) = \frac{{n}_{2}}{{λ}^{2}} exp ( \frac{- {n}_{2}^{2}}{2 {λ}^{2}} ) & \{ 2 \}\end{matrix}$$

wherein σ², λ² are mean square scattering amplitudes from a and b, respectively (See Wagner et al. 1983).

Assuming that the speckle noise on the ultrasound images is multiplicative, the noise can be modeled as:

aⱼ=n₁sⱼ {3}

bⱼ=n₂sⱼ {4}

wherein sⱼ is a true (noiseless) signal and j is a pixel within the block. Combining Eqn. {3} and {4} gives:

$$, \frac{{α}_{j}}{{b}_{j}} = \frac{{n}_{1}}{{n}_{2}} N , or {a}_{j} = {Nb}_{j} , \qquad \{ 5 \}$$

wherein: N=n₁/n₂, a division of two Rayleigh distributed variables.

Using the maximum likelihood method for parameter estimation, the matching TempBox to the template is found by maximizing the following conditional probability density function (pdf)

max_{d}p(a|b,d) {6}

wherein: d is a displacement vector, p(a|b,d) is a conditional probability, a is the vector containing all intensities in the template in frame t, and b is the vector containing all intensities in the TempBox in frame t+1.

Eqn. {6} states that the conditional probability is maximized when b is most like a, (i.e. a particular TempBox matches a Template). Since a and b are both vectors with j independent elements, the pdf in Eqn. {6} is equal to the multiplication of each single element's probability function. A probability function for a single element is calculated using the general Fundamental Theorem for any independent elements α and β (see for example, Papoulis and Pillai, Probability, random variables and stochastic processes with errata sheet, McGraw-Hill Science/Engineering/Math, 2001, pp. 130, 187, 236:

$${p}_{β} ( β ) = \frac{{p}_{α} ( α )}{\vert {g}^{′} ( α ) \vert} , \qquad \{ 7 \}$$

wherein: g(α) is a real solution to the random variable α's function β=g(α).

In the case of using RF envelope detected data, and using Eqn. {5} above,

g(N)=Nbⱼ, and |g′(N)|=bⱼ {8}

Using Eqn. {7}, the conditional pdf for one template and one TempBox in Eqn. {6} can be written as a product of single element pdf's:

$$p ( a \vert b , d ) = {\prod }_{j = 1}^{IJ} \frac{1}{{b}_{j}} {p}_{j} ( N ) \qquad \{ 9 \}$$

wherein: pⱼ(N) is the joint probability function of n₁ and n₂, i.e.,

$${p}_{j} ( \frac{{a}_{j}}{{b}_{j}} ) = {p}_{j} ( \frac{{n}_{1}}{{n}_{2}} ) ,$$

and IJ is the total number of pixels in the Template or TempBox.

Using Eqn. 6-15 (pp. 187) and solution to 6-59 (pp. 236) from Papoulis and Pillai (cited above), and Eqns. {1} and {2}, pⱼ(N) is found by evaluating the following integral:

$$\begin{matrix}\begin{matrix}{p}_{j} ( N ) = {\int }_{0}^{∞} {n}_{2} {p}_{1} ( {Nn}_{2} ) {p}_{2} ( {n}_{2} ) d {n}_{2} \\ = {\int }_{0}^{∞} {n}_{2} \{ \frac{{Nn}_{2}}{{σ}^{2}} exp ( \frac{- 1}{2 {σ}^{2}} {( N {n}_{2} )}^{2} ) \} \{ 11 \} \\ \{ \frac{{n}_{2}}{{λ}^{2}} exp ( \frac{- 1}{2 {λ}^{2}} {( {n}_{2} )}^{2} ) \} d {n}_{2} \\ = \frac{N}{{σ}^{2} {λ}^{2}} {\int }_{0}^{∞} {n}_{2}^{3} exp ( \frac{- {N}^{2} {λ}^{2} - {σ}^{2}}{2 {σ}^{2} {λ}^{2}} {( {n}_{2} )}^{2} ) d {n}_{2} \{ 12 \}\end{matrix} & \{ 10 \} \\ {p}_{j} ( N ) = \frac{{σ}^{2}}{{λ}^{2}} \frac{2 N}{{( {N}^{2} + \frac{{σ}^{2}}{{λ}^{2}} )}^{2}} & \{ 13 \}\end{matrix}$$

The last step uses integral number 3.381.4 from Gradshteyn and Ryzhik, Table of Integrals, Series and Products (2007). Assuming that σ=λ, then Eqn. {13} becomes:

$${p}_{j} ( N ) = \frac{2 N}{{( {N}^{2} + 1 )}^{2}} \qquad \{ 14 \}$$

Therefore the conditional pdf for RF-envelope-detected data in Eqn. {9} becomes:

$$\begin{matrix}p ( a \vert b , d ) = {\prod }_{j = 1}^{IJ} \frac{1}{{b}_{j}} {p}_{j} ( N ) \\ = \prod \frac{1}{{b}_{j}} \frac{2 N}{{( {N}^{2} + 1 )}^{2}} \\ = \prod \frac{1}{{b}_{j}} \frac{2 \frac{{a}_{j}}{{b}_{j}}}{{( \frac{{a}_{j}^{2}}{{b}_{j}^{2}} + 1 )}^{2}} \\ = {\prod }_{j = 1}^{IJ} \frac{2 {a}_{j}}{{( {a}_{j}^{2} + {b}_{j}^{2} )}^{2}}\end{matrix} \qquad \{ 15 \}$$

The maximization of Eqn. {15} is equivalent to the maximization of Eqn. {9}.

As previously described, the RF data undergoes a logarithmic compression in order to be displayed as a B-Scan image. Because most ultrasound machines do not offer access to RF signal, the compressed pixel intensities on the obtained B-Scan image must be accounted for. Because of this, Eqn. {5} becomes:

ln(aⱼ)=ln(N)+ln(bⱼ) {16}

Similar to the previous process with RF data:

g(N)=ln(N)+ln(bⱼ) {17}

### Thus,

$${g}^{′} ( N ) = \frac{1}{N} = \frac{{b}_{j}}{{a}_{j}} \qquad \{ 18 \}$$

Similar to the previous process for RF data, the conditional pdf of B-Scan data becomes:

$$\begin{matrix}p ( a \vert b , d ) = {\prod }_{j = 1}^{IJ} \frac{{a}_{j}}{{b}_{j}} {p}_{j} ( N ) \\ = \prod \frac{{a}_{j}}{{b}_{j}} \frac{2 N}{{( {N}^{2} + 1 )}^{2}} \\ = {\prod }_{j = 1}^{IJ} \frac{{a}_{j}}{{b}_{j}} \frac{2 \frac{{a}_{j}}{{b}_{j}}}{{( \frac{{a}_{j}^{2}}{{b}_{j}^{2}} + 1 )}^{2}}\end{matrix} \qquad \{ 19 \}$$

Let ãⱼ=ln(aⱼ), and let \tilde{b}ⱼ=ln(bⱼ), so that

$$\frac{{a}_{j}}{{b}_{j}} - exp ( {\tilde{a}}_{j} - {\tilde{b}}_{j} ) ,$$

wherein ãⱼ and \tilde{b}ⱼ are the vectors of B-Scan intensities in the Template and a single TempBox in frame t and t+1, respectively. Then Eqn. {19} becomes:

$$p ( \tilde{a} \vert \tilde{b} , d ) = {\prod }_{j = 1}^{ij} \frac{2 exp 2 ( {\tilde{a}}_{j} - {\tilde{b}}_{j} )}{{( exp ( 2 ( {\tilde{a}}_{j} - {\tilde{\tilde{b}}}_{j} ) ) + 1 )}^{2}} \qquad \{ 20 \}$$

The maximization of Eqn. {20} is equivalent to the maximization of Eqn. {6}. Eqn. {20} is a double exponential, and is considered an FT distribution.

It is often easier to compute the log-likelihood of Eqn. {20} instead of direct calculation. This is valid because logarithms are monotonically increasing, so that the logarithm of a function achieves the maximum at the same place as the function itself. Eqn. {20} then becomes the following objective function:

$$\begin{matrix}ln L = ln ( p ( \tilde{a} \vert \tilde{b} , d ) ) \\ = {\sum }_{j = 1}^{IJ} [ ln ( 2 ) + 2 ( {\tilde{a}}_{j} - {\tilde{b}}_{j} ) - 2 ln ( exp ( 2 ( {\tilde{a}}_{j} - {\tilde{b}}_{j} ) ) + 1 ) ]\end{matrix} \qquad \{ 21 \}$$

The maximization of Eqn. {21} is equivalent to the maximization of Eqn. {20}.

### Interframe Displacement Estimation

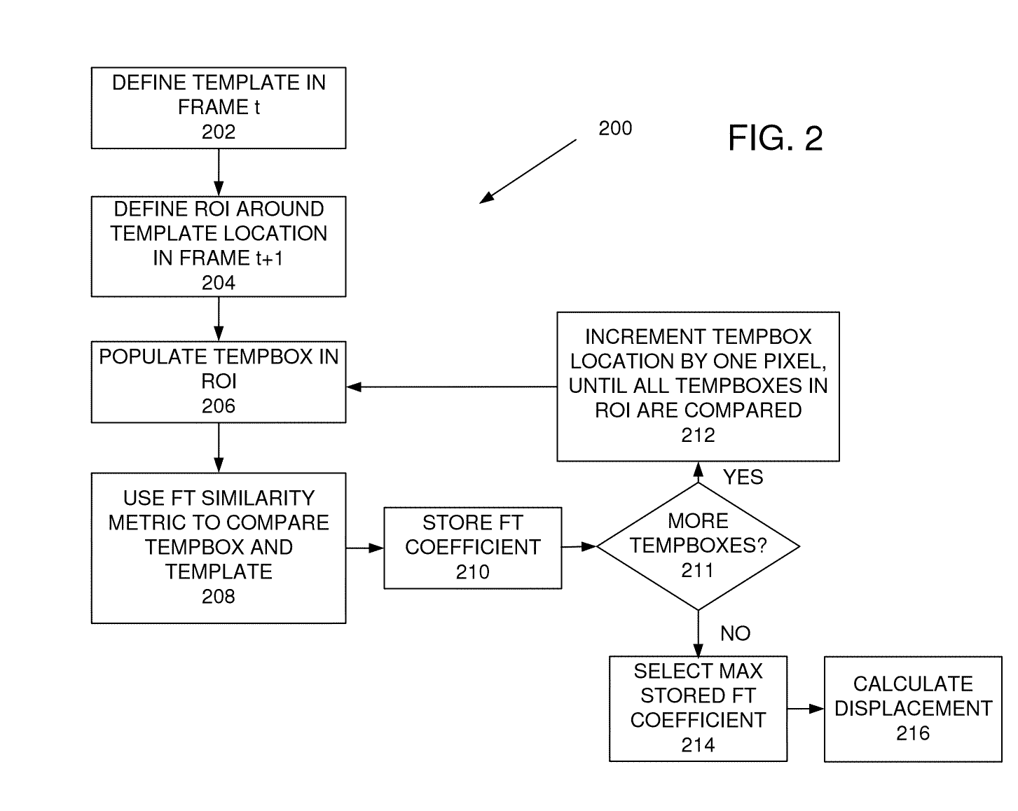

Calculation of interframe displacement between frame t and frame t+1 is shown in the flow chart of FIG. 2 that illustrates a representative interframe displacement method 200. At 202, a template of size I by J in frame t is defined. This template is a subsection of pixels in a frame t, as described above. At 204, an A by B ROI is defined in frame t+1, centered on the Template. At 206, a single TempBox, also of size I by J, is defined in the ROI in frame t+1. Next, at 208, a sum calculation such as that of Eqn. {21} is performed over all pixels in the template and a single TempBox in the ROI, giving a single FT likelihood coefficient that provides a comparison of the template and the TempBox. This FT coefficient is stored at 210. This is then repeated for all TempBoxes in the A by B ROI as determined at 211 by incrementing the TempBox location at 212 and repeating this calculation. In some examples, TempBox location is adjusted by one pixel until all TempBoxes in the ROI are compared. Typically, the TempBoxes are overlapping, and offset by one, two, or more pixels from each other. After repeating this process, there are A by B stored FT coefficients. The TempBox having the FT coefficient with the maximum value is considered a match, and is selected at 214. Based on the coordinates of the selected TempBox, the interframe displacement vector is calculated at 216. The interframe displacement vector d is calculated by subtracting the (x,z) location difference between the template and selected TempBox, i.e. d=(x₁−x₂, z₁−z₂), wherein x₂,z₂ is the location of the selected TempBox.

### Total Displacement Estimation

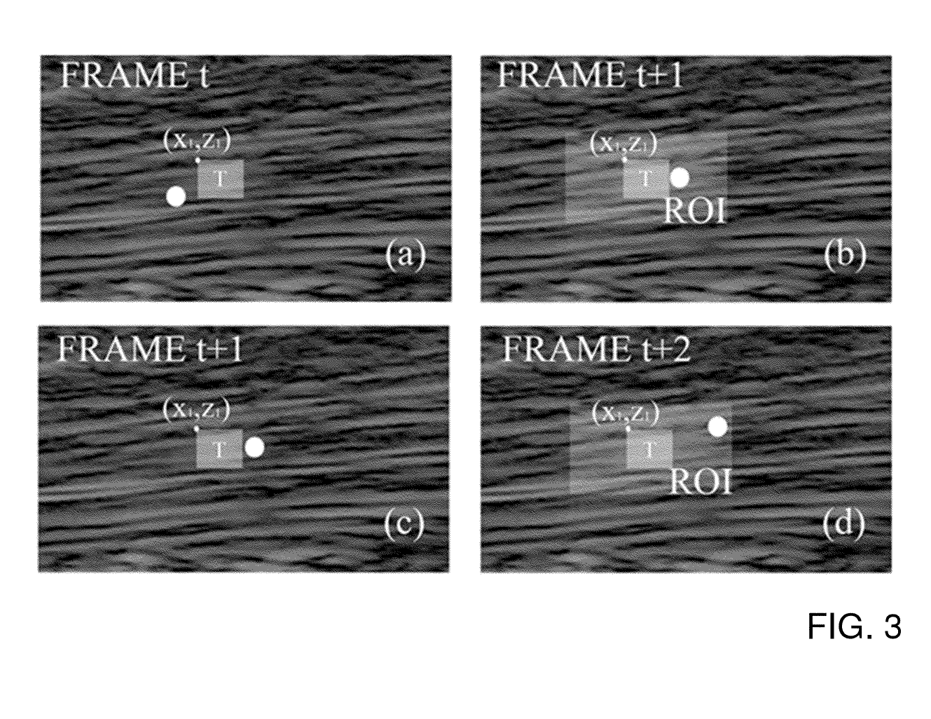

The determination of the total displacement of the MSK tissue excursion requires computation of the interframe displacement between all frames in the image sequence. This means that the interframe displacement between frame t and t+1 is first estimated, then between frame t+1 and t+2, and then between frame t+2 and t+3, and so on. The value for each interframe displacement between each set of frames is then cumulatively added to create a total displacement. In some disclosed methods, not all interframe displacements are calculated using a ROI that remains in the same position in the B-Scan image, referred to herein as a “fixed ROI.” This means that for the next two consecutive image frames, i.e. frame t+1 and frame t+2, the template block is updated with the data from frame t+1 at location x₁,z₁. This process can be visualized in FIG. 3 as a fixed ROI, whereby the displacement through the ROI located at x₁,z₁ is estimated using a stationary ROI. All other speckle tracking techniques work differently by tracking a specific location on the moving tissue itself (represented as a white circle in FIG. 3). This means their ROI changes position (follows the tissue) across the screen, during the B-Scan image sequence. As well, they use only the original template from their frame t for comparison to all subsequent image frames. However, in the disclosed methods, the ROI is stationary and the template always remains at location x₁,z₁. The template is updated for each new frame. This approach has a number of advantages: (a) If the B-Scan image has a small field-of-view, the entire MSK excursion can be estimated, and (b) if there was a tracking mis-match at some place in its displacement, the remaining displacement estimations would not suffer by compounding the error. This algorithm is in contrast to conventional speckle tracking algorithms which track the same location on the tendon as the tendon displaces across consecutive frames (i.e. the previous matching TempBox would become the new template for the next iteration). Therefore, tracking can be easily lost if the matching TempBox was actually incorrect, and then used as the next template.

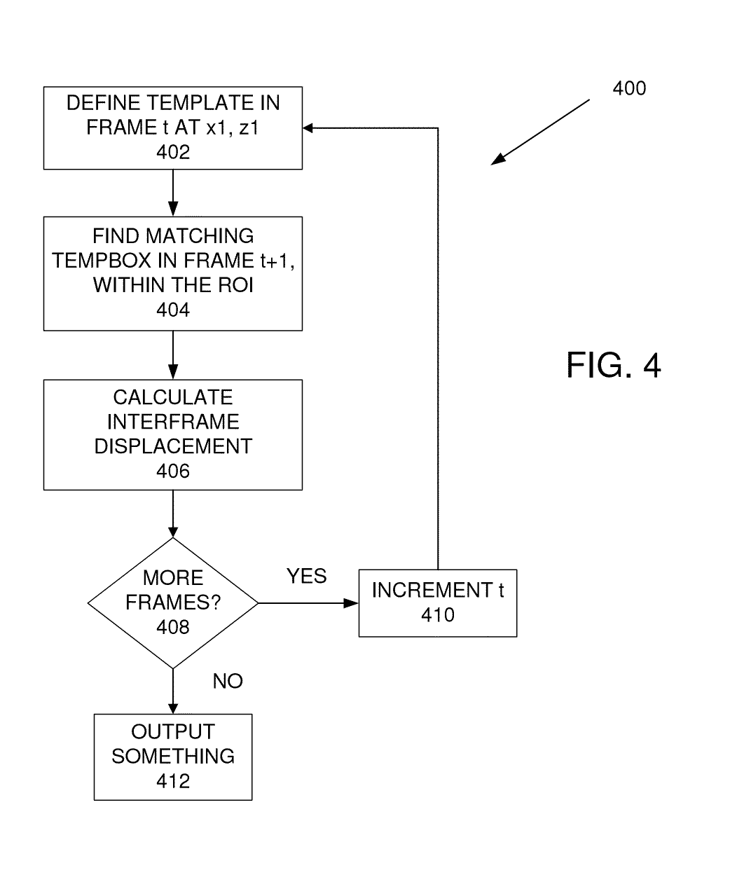

The flow chart in FIG. 4 describes a fixed ROI method 400. At 402, a template is defined in a frame t at coordinates x1, z1. At 404, a matching TempBox from a frame t+1 is found in the fixed ROI, and an associated interframe displacement is determined at 406. If additional frames are to be evaluated at determined at 408, then the frame identifier t is incremented at 410, and a TempBox in frame t+2 is identified and an associated displacement calculated. This process continues until no additional frames are selected, and a total displacement provided at 412 based on the interframe displacements.

Current commercially available ultrasound devices have limited MSK excursion tools available to clinicians or researchers. Some ultrasound machines have elastography tools which estimate MSK displacement fields in order to display the tissue strain. A displacement field is a vectoral representation quantifying the magnitude of total displacement at many different locations on the MSK tissue. Usually, the displacement field data is hidden from user, but the machine will display various strain measurements as a color map. The disclosed technology allows access to total displacement, incremental velocity and incremental displacement. This means that the user can estimate the displacement and velocity at any point in the MSK excursion. This is not currently available on commercial systems. Additionally some machines have a Tissue Doppler Imaging (TDI) function to estimate tissue motion. This function is mostly used for echocardiography, and has limited use for MSK excursion. In contrast to commercially available tools, the disclosed methods can be used with open-ended ultrasound machines with a research interface, or on a PC by simply exporting the ultrasound movie file. The user does not require a different ultrasound machine, or expensive software “add-ons” from a manufacturer.

When referring to the displacement methods itself, some advantages of using the disclosed methods include: using a similarity measure accounting for data compression, having a fixed ROI and template location for searching, incremental tracking, and real time algorithms catered specifically to MSK displacement.

The success of speckle tracking is highly dependent on parameters such as the ultrasound system's frame rate, the frequency of the transducer, the similarity measure chosen, the tissue velocity, and the template (kernel) size and search region, to name a few. Also, speckle tracking in 2D B-Scan videos can be computationally intensive, and hence better techniques are needed to implement it on lower-cost, mid-range ultrasound systems. Therefore, no two tracking algorithms are alike, and algorithms can be tailored for specific ultrasound machines. In some examples, the disclosed methods and apparatus are based on some or all of the following features, or exhibit certain listed advantages:
1. Fisher-Tippett is used as a similarity measure to represent the speckle characteristics in B-Scan images. Logarithmic compression on the displayed B-Scan images is accounted for.
2. A single fixed ROI search technique is used to track large displacements, and to lessen the effects of errors that cause tracking loss. The previously published literature uses a NCC-multi-kernel system along with a multiple gating technique. Gating is used mainly for two reasons: (1) to overcome tracking loss due from speckle decorrelation, and (2) track large displacements. A single ROI searching technique provides better computational efficiency in comparison with a multi-ROI. The fixed ROI technique contrasts with many existing algorithms in which the same piece of the tendon is tracked across the B-Scan.
3. Use of an incremental tracking algorithm that tracks interframe displacement over a sequence of images. Also, a kernel for the first image frame is not compared to all subsequent image frames. For a given image frame k, the kernel is established and then used on the consecutive frame, k+1. Once the inter-frame displacement is determined, a new kernel is then established in frame k+1, and the consecutive frame k+2 is compared to find the inter-frame displacement. This way, even ultrasound machines with low frame rates (20 frames-per-second) can be used.
4. The techniques can be performed in real time.
5. The methods can be applied to tracking Musculoskeletal displacement in two dimensions (axial and lateral), using 2D B-Scan Ultrasound images
6. MSK excursion estimations are possible on closed-commercial grade ultrasound systems, by tracking the MSK motion on an exported ultrasound movie file on a PC. Therefore, the disclosed methods provide a cost effective solution, because the clinician or researcher can use existing ultrasound hardware.

### Template Selection

The above methods and apparatus permit speckle tracking for use in applications such as estimation of tendon displacements. Successful implementation of these speckle tracking algorithms depends on many parameters. For the disclosed methods, such parameters include the location of the template, the size of the template, the frame rate of the ultrasound machine, and the searching strategy. It is difficult for an ultrasound operator (clinician) to preselect these parameters in advance. Suitable parameter settings can be obtained from analysis of prior studies so as to permit automatic parameter selection technique and optimal tissue tracking.

### Template Auto-Location

The template is preferably located on the tendon in an ultrasound image sequence at a location that permits superior tracking. The ultrasound image sequence may be a B-Scan image sequence or an RF image sequence. Misalignment of the template with respect to the tendon will affect the tracking performance. An operator may select a poor location for the template, or even with an initial good template location, the tendon may shift laterally during the image sequence. Thus, the template may not remain on the tendon for the entire excursion when using a stationary ROI technique. In addition, there may be regions in the ultrasound image sequences that have enhancement or shadow artifacts, thus total displacement estimations are not consistent at all locations along the tendon. It is possible to observe the total displacement of tissue at all or many points in the image field of view using a so-called displacement field. In order to create a displacement field, the cumulative displacement methods discussed above can be used. The template location is varied, by starting at an initial location in ultrasound image frames, for example in a top left location. This gives an estimate of the total displacement of the tissue at that point. Afterwards, this process is repeated one or more other locations, giving additional total displacement estimates at these locations. Typically, many (or all) available locations are used to provide corresponding displacement estimates that define a displacement field. This displacement field represents estimated displacement at a given location on the tissue within the ultrasound image field of view, including all points on the tendon's entire excursion. A displacement field can be graphically illustrated as a two dimensional view of a three dimensional color map, wherein some or all locations in an x-z plane are associated with a displacement magnitude and total displacement at each x-z point shown as a color or gray-scale value. Displacement field direction can be similarly represented.

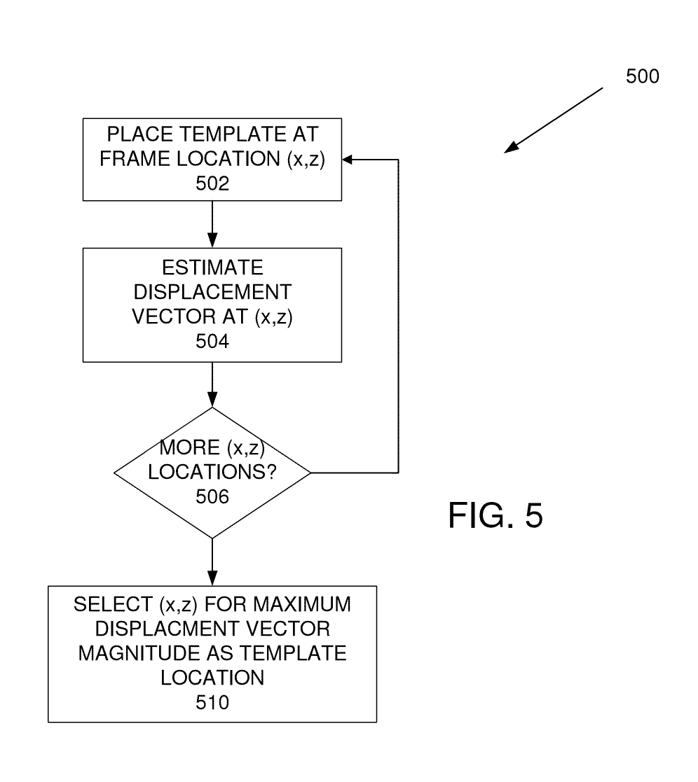

A representative method of establishing a displacement field is illustrated in FIG. 5. At 502, a template is situated in a frame at a location defined by coordinates (x, z) and at 504 a displacement vector (or magnitude or direction) is determined with respect to a subsequent frame. If displacement field values are to be determined for additional locations at determined at 506, the template is placed at new location at 502 and the displacement vector estimated at 504. If all frame locations of interest have been evaluated, coordinates associated with a maximum displacement vector magnitude are assigned as a template location at 510. In some examples, displacement vector magnitude, direction, or a combination thereof can be used to establish a template location.

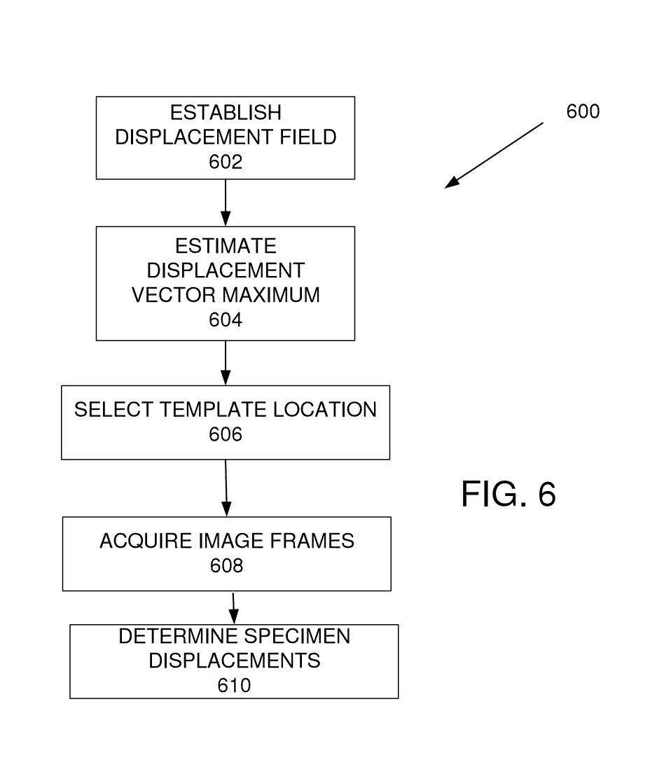

A representative method 600 of speckle tracking using a displacement field is illustrated in FIG. 6. At 602, a displacement field is created based on some or all points in an image field of view, for an entire image sequence or a portion thereof. The displacement field can be determined in a scan-line approach that evaluates image field points in a raster-scanning pattern can be used to evaluate total displacement at all x, z locations within the image field of view. To reduce numbers of computations, x, z locations can be incremented in multiples of two, three, four, or more, to create a sparse displacement field that lacks displacement vectors associated with some points in the image field of view. Other selected sets of points in the image field can be used such as random image points or other arrangements of points.

At 604, a maximum displacement value in the displacement field is determined, and the corresponding location in the image field is selected at 606 as a template location. Since the tendon lies somewhere within this ultrasound image field of view, and since it moves more than any other type of tissue, the maximum displacement value found corresponds to the best location to place the template to track the tendon. This location is defined as the ‘ideal’ template location, but other locations can be used. The ultrasound transducer head is generally secured with respect to a subject and does not move significantly relative to the tissue it is imaging, and the ideal (or other identified) template location can be used for subsequent tendon tracking. Therefore, this localization procedure serves as a calibration step used to determine an ideal template location after placing the transducer onto the body, such as onto a wrist, knee, elbow, finger or other location. With this approach, the template location can be determined without guesswork and without time consuming trial and error. At 608, image frames are acquired, and at 610, specimen displacements are determined using the selected template location.

### Template Size

The size of the template chosen in frame t can affect the success of tracking. For instance, if the template is too large, regions of non-uniform motion can be included. This tends to result in an averaging of the displacement estimation due to the inclusion of non-tendon tissue within the template. If the template is too small, associated displacement estimates are susceptible to noise and can cause ambiguity and mismatch. Furthermore, a small template can contribute to an aperture problem if the tendon image has large regions (spots) of uniform grayscale intensity in B-Scan, or uniform RF values. In such cases, as the tendon displaces across the ultrasound image field of view, it moves through the ROI centered on the template. If the template is smaller than the uniform grayscale (value) spots, the tendon appears to be stationary. Typically, template sizes that are about 50-to-70% of tendon thickness (measured laterally to tendon length) are preferred. To find the template size, the displacement field (as described in the template auto-location technique above) is used. Applying an image segmentation procedure to the displacement field, the tendon width can be estimated, and a suitable template size selected, typically about 10%, 20%, 30%, 40%, 50%, 60%, 70%, or 80% of the tendon width. One or both of displacement field magnitude and direction can be used in the image segmentation.

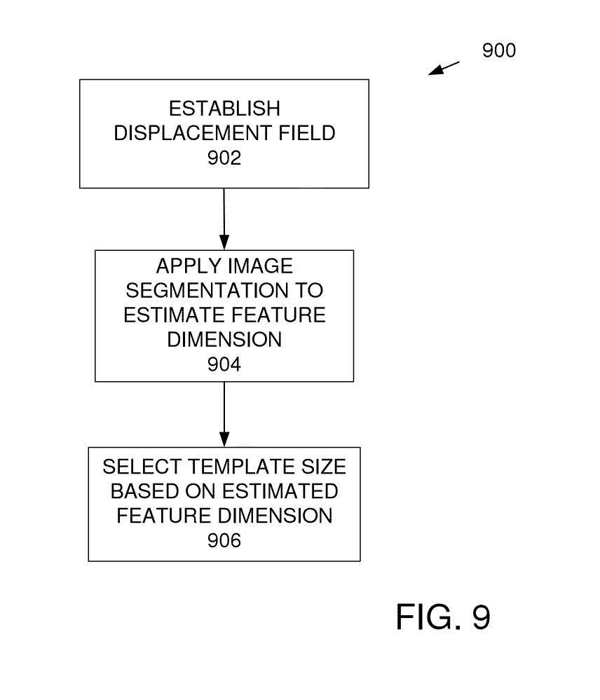

A representative method 900 of establishing a template size, or one or more dimensions of a template region is shown in FIG. 9. At 902, a displacement field is determined, and typically a displacement magnitude associated with the displacement field. One or more image segmentation procedures are applied to the displacement field (or the associated magnitudes) at 904. Segmentation procedures permit identification of a feature of interest, and one or more dimensions of the feature of interest. For example, a tendon width can be estimated based on an image segmentation process that distinguishes image or frame portions associated with relatively large frame-to-frame displacements. At 906, a template size or one or more dimensions can be selected based on the estimated dimension of the feature of interest. Typically, a template size (length and width) is selected to correspond to about 40% to 80% of the estimated feature dimension. The method 900 requires no operator assistance—specimen images can be automatically processed to determine template size, if desired.

### Frame Skipping Auto-Select

Not all frames need to be compared in determining a displacement field, and a suitable number of frames and frame rate can be dependent on imaging system details. Image sequence frame rate (number of frames per second) and tendon velocity (displacement/second) are typically important considerations in speckle tracking. Since every ultrasound imaging system is different, image resolution may not be sufficient to detect small interframe displacements. This is a function of system frame rate and lateral resolution, as well as the tendon lateral displacement and velocity. In particular, a tendon velocity must not be too fast with respect to image frame rate, or tracking can be lost. For fast moving tendons, the frame rate of the ultrasound image capture must be high enough, to capture image sequences with reasonable displacements between frames. If the interframe displacement were too high and were captured with a low frame rate, speckle decorrelation can occur, causing matching errors for the tracking algorithm. Conversely, if the interframe displacement was low and the frame rate was high, it may be difficult to capture any motion between consecutive frames. A representative method of estimating a suitable interframe displacement can mitigate these problems by skipping frames when comparing the template to potential blocks in the ROI, i.e., by comparing the template in frame t to the blocks in frame t+k, wherein k is an integer. This approach is based on the assumption that the speckle does not decorrelate too much between frames t and t+k and that the velocity is constant (the displacement is linear) in the interval between frames t and t+k.

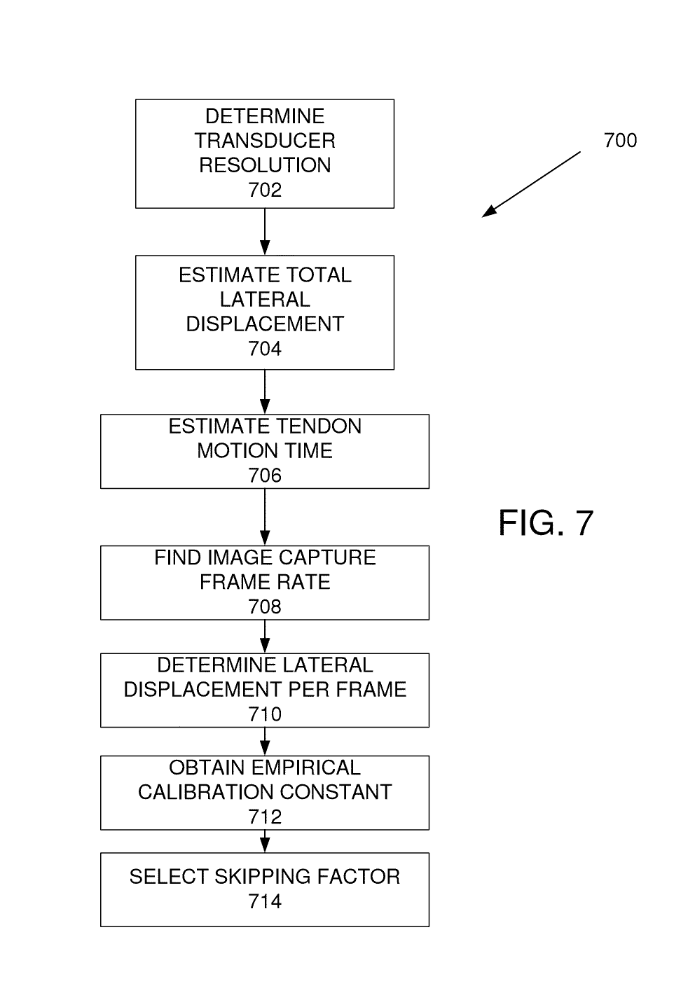

A representative method 700 of determining a suitable frame skipping number k is shown in FIG. 7. Disclosed herein is a representative method 700 in terms of transducer lateral resolution, an expected lateral displacement per frame, and an empirical constant γ. At 702, transducer lateral resolution R_{L}, can be obtained by a calibration of the ultrasound transducer used for image capture, in which an object of known dimensions is placed between gel pads under the transducer, at the approximate depth of the tissue to be imaged. This way, the mm/pixel ratio can be estimated, thereby providing a value for R_{L}. This calibration would only have to be done once for a particular transducer.

At 704, an expected lateral displacement per frame & can be determined as follows. Using the displacement field (as described above), an expected total lateral displacement, d_{T} is found, which corresponds to the maximum value in the displacement field. At 706, a total time t of tendon motion is found. This can be done by finding the number of frames containing tendon motion, by frame-to-frame analysis of the image sequence at the x, y point corresponding to maximum displacement, when there is zero interframe displacement at that point. This will occur just prior to the beginning of tendon motion, and just after the end of tendon motion. At 708, the image capture frame rate FR of the ultrasound machine's hardware is found, which is well known and usually contained within the image sequence file header. The FR and the total number of frames containing motion can be used to find the displacement time T. At 710, an estimate of the expected lateral displacement per frame ε can be calculated as follows:

$$ɛ = \frac{{d}_{T}}{T} \cdot \frac{1}{FR} \qquad \{ 22 \}$$

wherein d_{T} is the expected total displacement, T is the total time of displacement, and FR is the system's frame rate. The expected lateral displacement per frame ε is typically in units of mm/frame or other units of length per frame.

At 712, an empirical calibration constant γ is determined. If the lateral resolution is coarse, and ε is small, the speckle tracking algorithm may not be able to detect any interframe displacement. Therefore, by comparing alternate frames, such as frames t and t+k, the expected lateral displacement in k frames becomes k·ε. Therefore, γ, can be defined as:

$$\frac{k \cdot ɛ}{{R}_{L}} ≅ γ \qquad \{ 23 \}$$

wherein k is the frame skipping number, ε is the expected lateral displacement per frame, and R_{L} is the lateral resolution. A suitable value of γ generally has a value of about 8.24 pixels. Rearranging Eqn. {23} and using the empirically derived γ constant of 8.24 pixels, an ideal frame skipping number for subsequent data sets is estimated at 714 as:

$$k ≅ \frac{γ {R}_{L}}{ɛ} . \qquad \{ 24 \}$$

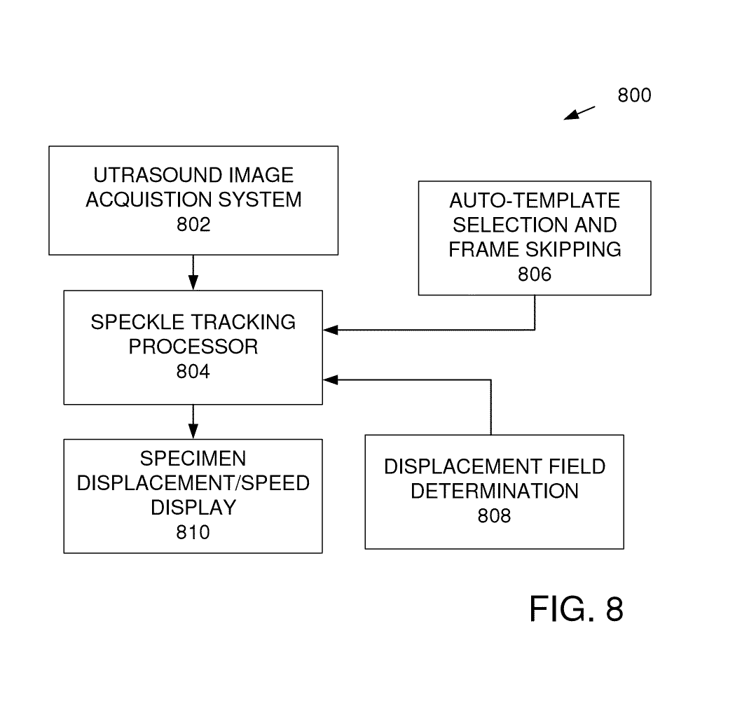

A representative tissue tracking apparatus 800 is illustrated in FIG. 8. An ultrasound image acquisition system 802 is coupled to a speckle tracking processor 804. The processor 804 is coupled to one or more computer readable media (or a network connection) so as to receive computer-executable instructions 806, 808 for auto selection of template size and location, and a frame skipping number as well as instructions for determining a displacement field. The processor 804 determines tissue displacements based on comparisons of a template region and test regions (TempBoxes) in series of images. Specimen displacement or speeds are provided at an output device 810 such as a display device, or results are coupled to a network. The processor 804 can be distinct from the acquisition system 802, or be a separate processor. In some examples, the processor 804 can be located or a network or be otherwise remote.

Having described and illustrated the principles of the disclosed technology with reference to the illustrated embodiments, it will be recognized that the illustrated embodiments can be modified in arrangement and detail without departing from such principles. For instance, elements of the illustrated embodiments shown in software may be implemented in hardware and vice-versa. Also, the technologies from any example can be combined with the technologies described in any one or more of the other examples. It will be appreciated that procedures and functions such as those described with reference to the illustrated examples can be implemented in a single hardware or software module, or separate modules can be provided. The particular arrangements above are provided for convenient illustration, and other arrangements can be used.

We claim:

1. A method of estimating a tissue displacement, comprising:
selecting a template region in a first ultrasound image of a region of interest, wherein the first ultrasound image exhibits speckle;
comparing a plurality of image portions in a second ultrasound image of the region of interest to the template region, wherein the second ultrasound image exhibits speckle; and
based on the comparisons, estimating a tissue displacement.

2. The method of claim 1, wherein the comparisons are based on a Fisher-Tippet distribution or a Rayleigh distribution.

3. The method of claim 1, wherein the first and second images are B-scan images, and further comprising establishing a total tissue displacement based on comparisons of image portions of a series of B-scan images to the template region.

4. The method of claim 1, wherein the first and second images are RF envelope images, and further comprising establishing a total tissue displacement based on comparisons of image portions of a series of RF envelope images to the template region.

5. The method of claim 1, further comprising determining a template region location based on a displacement field associated with at least two ultrasound images.

6. The method of claim 1, wherein the second ultrasound image is the next image with respect to the first image.

7. The method of claim 1, wherein at least one or more ultrasound images are obtained prior to the second ultrasound image.

8. The method of claim 7, further comprising determining a skip factor associated with a number of images between the first ultrasound image and the second ultrasound image.

9. The method of claim 1, further comprising selecting a template region sized based on an estimated image to image displacement and an image acquisition rate.

10. An apparatus, comprising:
a memory configured to store a plurality of ultrasound images; and
a processor that receives the images from the memory, selects a region of interest and a template region in a first image, compares image portions in each of the series of images with the template region, and provides a tissue displacement based on the comparison.

11. The apparatus of claim 10, wherein the processor establishes the comparison based on a Fisher-Tippet distribution.

12. The apparatus of claim 11, wherein the processor establishes the comparison based on image values corresponding to logarithmic functions of scattering amplitudes.

13. The apparatus of claim 10, wherein the images are B-scan images.

14. The apparatus of claim 10, wherein the processor sequentially compares image portions in the series of images.

15. The apparatus of claim 10, wherein the processor compares images in the series of images based on a skipping number associated with a number of images to be skipped between comparisons.

16. The apparatus of claim 15, wherein the processor determines the skipping number based on an expected lateral displacement per sequential image and a lateral resolution.

17. The apparatus of claim 15, wherein the processor performs image segmentation on at least one image to identify a specimen feature of interest, and determines a template region dimension based on a dimension of the specimen feature of interest in the at least one image.

18. The apparatus of claim 17, wherein the template region dimension is between about 30% and 80% of the specimen feature dimension.

19. The apparatus of claim 18, wherein the specimen feature of interest is a tendon.

20. The apparatus of claim 10, wherein the processor provides the comparison based on maximization of
$$p ( \tilde{a} \vert \tilde{b} , \tilde{d} ) = {\prod }_{j = 1}^{IJ} \frac{2 exp 2 ( {\tilde{a}}_{j} - {\tilde{b}}_{j} )}{{[ exp 2 ( {\tilde{a}}_{j} - {\tilde{b}}_{j} ) + 1 ]}^{2}} ,$$
wherein and \tilde{b}ⱼ are elements of vectors of B-Scan intensities in the template region and a series of image regions in each of the series of images.

21. The apparatus of claim 10, wherein the processor provides the comparison based on a Fisher-Tippet distribution or a Rayleigh distribution.

22. At least one computer readable medium containing computer-executable instructions for performing a method comprising:
defining a template region in a selected image frame based on an image resolution, a specimen displacement between the selected image frame and an adjacent image frame, and an image feature size;
comparing an image portion in the template region in the selected image frame with a plurality of test regions in a different image frame; and
based on the comparison, estimating an image feature displacement.

23. The at least one computer readable medium of claim 22, wherein the comparison is based on a Fisher-Tippet distribution.

24. A method, comprising:
obtaining at least a first ultrasound image and a second ultrasound image of a specimen, wherein the first and second ultrasound images exhibit speckle;
establishing at least a portion of a displacement field based on the first and second ultrasound images;
determining a specimen feature dimension by applying image segmentation to the displacement field; and
based on the specimen feature dimension determined by the image segmentation of the displacement field, selecting a size of a template region.

25. The method of claim 24, further comprising obtaining a plurality of ultrasound images exhibiting speckle, and processing the plurality of ultrasound images exhibiting speckle based on comparisons of test regions in the plurality of ultrasound images with respect to the template region.

26. The method of claim 25, wherein the plurality of specimen images is processed to determine image feature displacements or image feature speeds.
